(19) United States
(12) Patent Application Publication
(10) Pub. No.: US 2011/0152755 A1
(43) Pub. Date: Jun. 23, 2011

(54) Device for injecting solid matter

(75) Inventor: Christian SCHMALZ, Lyss (CH)

(73) Assignee: FORTEQ NIDAU AG, Nidau (CH)

(21) Appl. No.: 12/973,490

(22) Filed: Dec. 20, 2010

(30) Foreign Application Priority Data
Dec. 21, 2009 (EP) 09405230.5

(51) Int. Cl. A61M 5/315

(52) U.S. Cl. 604/60

(57) ABSTRACT

A device (1; 1001) for injecting solid matter (500; 1500) into a human or animal body (600; 1600) comprises a housing (100; 1100), a syringe body (200; 1200) for holding the solid matter (500; 1500), connected to a cannula (220; 1220), a primary plunger (230; 1230), which can be displaced in the syringe body (200; 1200) and in the cannula (220; 1220), and a secondary plunger (300; 1300), which can be displaced in the housing (100; 1100). The device (1; 1001) comprises a force-transmission mechanism, which can act between the secondary plunger (300; 1300) and the syringe body (200; 1200) and/or the cannula (220; 1220), wherein an actuation of the secondary plunger (300; 1300) in the distal direction can bring about a displacement of the syringe body (200; 1200) and/or the cannula (220; 1220) in the proximal direction.

### TECHNICAL FIELD

The invention relates to a device for injecting solid matter into a human or animal body, comprising a housing, a syringe body for holding the solid matter, the syringe body being connected to a cannula, a primary plunger, which can be displaced in the syringe body and in the cannula, and a secondary plunger, which can be displaced in the housing.

### PRIOR ART

The injection of solid matter is a conventional method for long-term medication (depot injection). To this end, a depot is placed into the body and it dispenses the medicament to the body over a predetermined period, ideally in a constant and continuous fashion. Typical applications of long-term medication are pain therapies, hormone therapies for contraceptive purposes, etc. Furthermore, the injection of solid matter is also applied in conjunction with subcutaneous injections of chips, e.g. microprocessors or storage chips.

Solid matter is typically injected by virtue of the fact that the solid matter is held in a cannula situated in the body by means of a plunger, whereupon the cannula is withdrawn with the plunger remaining in place, and thus leaves the solid matter in the body.

US 2009/0209903 A1 (S.C.R.A.S) shows such an injection device for intramuscular or subcutaneous injections of solid or semi-solid implants. The device comprises a main body, to which a hollow needle is connected, and a second body arranged coaxially in the main body and surrounding the hollow needle, and a plunger, which can be inserted coaxially into the hollow needle. The plunger can be blocked in the second body. During use, the main body is guided downwards, as a result of which the hollow needle penetrates the skin, with the plunger being carried along at the same time. The thumb presses the second body against the skin, while the index and middle fingers grasp the main body and withdraw it in the proximal direction, as a result of which the main body is moved upwards along the second body, together with the hollow needle but without the plunger. Once the hollow needle has been guided out of the body, the plunger is latched into the main body and guided out of the body.

The disadvantage of known devices for injecting solid matter is that their operation is unintuitive. More particularly, a plurality of different actions typically have to be performed, such as inserting the plunger, withdrawing the cannula without the plunger, withdrawing the plunger, etc. This is a considerable challenge for novice users of such devices. Furthermore, improper handling of such a device can cause further pain to the person or animal to be injected in addition to that caused by the introduction of the cannula.

Up until now, the user had to keep the plunger secure in its position after the cannula was inserted into the body and after the solid matter was placed into the cannula, and withdraw the cannula at the same time. This movement is problematic or difficult to perform inasmuch as the device does not have a stop in respect of proximal movement, which stop would prevent the primary plunger from being withdrawn at the same time as the syringe body and/or the cannula, as a result of which the solid matter may under certain circumstances not remain in the envisaged space thereof. This was only prevented by virtue of the fact that the user has held the plunger fixed in its position relative to the body. However, a human or an animal typically does not hold still during the injection. The unusual procedure of inserting solid matter into the tissue alone often leads to tension in humans and, possibly, to uncontrolled movements such as flinching or shivering. Additionally, the human or the animal also feels pain when the cannula is inserted under the skin, in particular if local anesthetization of the puncture site is dispensed with, which is a further reason for uncontrolled movements. These uncontrolled movements lead to problems for the user during the injection because they have to mirror the movements of the body in parallel and synchronous with the plunger and at the same time have to withdraw the cannula.

### SUMMARY OF THE INVENTION

An object of the invention is to develop a device that is part of the technical field mentioned at the outset for injecting solid matter and is simple, intuitive and safe to operate, and additionally has a cost-effective and simple design.

The object is achieved by the features of claim 1. According to the invention, the device comprises a force-transmission mechanism, which can act between the secondary plunger and the syringe body and/or the cannula, wherein an actuation of the secondary plunger in the distal direction can bring about a displacement of the syringe body and/or the cannula in the proximal direction.

In the following text, the term “distal” refers to a direction towards the body and the term “proximal” refers to the direction opposite thereto, or away from the body. In relation to the device, the two directions should respectively be understood as being parallel to a longitudinal axis, wherein the longitudinal axis can for example be defined by the housing, the syringe body, the cannula or by one of the two plungers. Unless denoted otherwise, the directions “distal” and “proximal” should be understood as being parallel to a longitudinal axis of the primary plunger. During the application of the device, the cannula at least for some of the time protrudes out of the housing in the distal direction.

After the cannula is guided into the body and the solid matter has been placed into the cannula by the primary plunger, the syringe body and/or the cannula are typically withdrawn while the primary plunger is fixed in its position with respect to the body such that the solid matter remains in the body. The force-transmission mechanism between the secondary plunger and the syringe body now creates a device by means of which the syringe body and/or the cannula can be displaced in the proximal direction by sliding the secondary plunger in the distal direction. This brings about a particularly ergonomic operation of the device because when the cannula is in the body, more particularly if the cannula is completely in the body up to the stop on the housing, uncontrolled movements of the body can be absorbed effortlessly by light pressure in the distal direction, more particularly by inserting the secondary plunger, and so the desired position of the solid matter can be maintained. Moreover, this allows the withdrawal of the cannula to be brought about by the ergonomic and intuitively preferred distal movement of the secondary plunger.

The housing preferably comprises holding elements, particularly in a proximal region. This allows the device to be held like a conventional syringe, namely by the holding elements being guided by index and middle fingers, and the plunger, more particularly the secondary plunger, being guided by the thumb. The holding elements can be designed as two opposing elements, radially protruding outwards, or as an encircling annulus. A person skilled in the art knows of further options for implementing such holding elements in an ergonomic fashion.

The device preferably comprises a rack-and-pinion gear. The rack-and-pinion gear can be installed into the device such that a distal movement of the secondary plunger can be converted into a proximal movement of the syringe body and/or the cannula, and this brings about a possible implementation of the force-transmission mechanism. The advantage of this is that the device can be operated like a conventional syringe, without an element of the device having to be guided in the distal direction, which is unfamiliar to the user, in order to withdraw the syringe body and/or the cannula. This allows the relative movement between the syringe body (and/or the cannula) and the secondary plunger to be implemented in a particularly simple fashion, in particular because toothed racks and gearwheels can be produced cost-effectively and easily, and are simple to install. Moreover, a rack-and-pinion gear ensures that the movements of the secondary plunger and the syringe body and/or the cannula are clearly defined, as a result of which operation of the device is further simplified and the susceptibility to faults is further reduced.

Alternatively, a rack-and-pinion gear may also be dispensed with, with the force transmission being implemented differently (see below).

The force-transmission mechanism is preferably embodied as a rack-and-pinion gear, which comprises a first toothed rack, a second toothed rack and a gearwheel, wherein
a) the gearwheel is mounted in the housing such that it can rotate freely; and
b) the first toothed rack is connected to the syringe body; and
c) the second toothed rack is connected to the secondary plunger; wherein
d) the gearwheel interacts with the first and the second toothed rack.

To this end, the first toothed rack and the syringe body and/or the second toothed rack and the secondary plunger can each have an integral design. This brings about a cost-effective and simple design of the device. The gearwheel is preferably produced separately, as an individual element. By way of example, the gearwheel is mounted on a pin, which is fixedly connected to the housing, wherein, for example, provision can be made for a clip-on connection for attaching the gearwheel on the pin, which is known to a person skilled in the art. The pin can preferably also be integrally connected to the housing in order to continue to achieve a simple design of the device.

The force-transmission mechanism can alternatively also have a different design. By way of example, provision could also be made for a toothless wheel instead of the gearwheel and the toothed racks, which toothless wheel can interact with appropriate regions of the secondary plunger and the syringe body by friction, with, however, a certain surface pressure having to be ensured between the toothless wheel and the corresponding regions. Furthermore, a connection of the syringe body and/or the cannula to the secondary plunger via a deflection element can also serve as force-transmission mechanism. To this end, the deflection element can be provided in a proximal region of the housing and be connected to the latter. A pin or a roller can be provided at right angles to the longitudinal direction of the device for the deflection. In order to transmit the movement, use can be made of a thread, a wire, a band or the like, which is connected to the syringe body or the cannula at a first end and to the secondary plunger at a second end and is deflected proximally about the pin or the roller. Finally, the cannula can also be withdrawn using a spring, with the cannula protruding from the housing when the spring is tensioned and only partly protrudes from the housing, more particularly not at all, if the spring is relaxed. This spring would be tensioned when the secondary plunger is inserted and relaxed in order to withdraw the cannula and thus constitutes a further option for a force-transmission mechanism. During such a withdrawal, there should optionally be prevention in place to stop the cannula being withdrawn from the body too rapidly after overcoming static friction therein. To this end, provision could be made for a brake, by means of which the withdrawal velocity could be controlled. By way of example, a resilient element, which is arranged laterally on the housing and can clamp the syringe body when pressure is applied by a finger, is a feasible implementation of such a brake. A person skilled in the art is also aware of further options for implementing a simple implementation of the proximal withdrawal of the cannula.

The device preferably comprises a first locking device, by means of which the secondary plunger can be blocked in respect of an axial displaceability in the housing. The cannula is preferably inserted into the body at first and the solid matter is subsequently guided into the cannula via the primary plunger. Only once the solid matter has assumed the position provided in the cannula, more particularly in a region of the cannula that protrudes from the housing, is the first locking device unlocked, which allows the insertion of the secondary plunger. This prevents inadvertent displacement of the secondary plunger, more particularly this prevents the syringe body and/or the cannula from being withdrawn before the solid matter has been placed into the cannula. This is because if the primary plunger has not been advanced enough but the secondary plunger could nevertheless be actuated, there is the risk of the primary plunger and the solid matter being displaced distally and the cannula being displaced proximally at the same time, as a result of which the typically blunt solid matter would be forced into the body, causing significant pain to the human or animal receiving the injection. The first locking device preferably comprises a resilient element connected to the housing and a recess in the secondary plunger, with the resilient element being able to engage with the recess. The resilient element preferably engages with the recess in the relaxed state and a force has to be actuated thereon if it should be moved from the recess.

Alternatively, the first locking device can also have a different design or the first locking device can be dispensed with completely. In the latter case, the user must take care to ensure that the secondary plunger should only be distally inserted into the housing after the primary plunger is completely inserted in the distal direction.

The first locking device can preferably be deactivated by the primary plunger, more particularly by a distal displacement of the primary plunger. The locking device is preferably deactivated after the complete distal displacement of the primary plunger, i.e. after the solid matter has been placed into the cannula. This brings about particularly simple operation of the device. This is because the primary plunger is preferably displaced completely through the syringe body in the distal direction and into the cannula before the secondary plunger is actuated. This is because the cannula should only be withdrawn after the solid matter has been placed therein. Thus, deactivating the first locking device in some respects automatically coordinates a preferred procedure in sequence, and so the syringe body and/or the cannula cannot inadvertently be withdrawn before the solid matter is introduced. To this end, the plunger head of the primary plunger is preferably designed such that it can be used to lift the resilient element from the recess of the secondary plunger.

The first locking device can alternatively also be deactivated in a different fashion. By way of example, the deactivation could be carried out directly by hand after the primary plunger has transferred the solid matter into the cannula. The deactivation can for example be brought about by a bar that can be inserted radially or in another manner known to a person skilled in the art.

The device preferably comprises a first energy store, wherein energy from the first energy store can be used for the distal displacement of the primary plunger. This allows the user to place the solid matter into the cannula in a particularly simple fashion without themselves having to expend energy apart from activating the energy store. Furthermore, the user cannot control where the solid matter is in the cannula, which can result in certain uncertainties during use. Embodying a first energy store for actuating the primary plunger brings about automatic introduction of the solid matter into the cannula, which can take the aforementioned uncertainties from the user. This is because the solid matter is typically placed in the cannula after the cannula has been introduced into the body. Hence, the use of a first energy store is also advantageous in that the solid matter can thereby be placed into the cannula in a relatively rapid fashion, as a result of which the duration of the injection, more particularly the amount of time that the cannula is in the body, can be reduced. These are obvious concessions to the person or animal to be injected. It goes without saying that the device can also be provided for distal displacement of the primary plunger before the cannula is inserted into the body. To prevent solid matter from falling out of the cannula in this case, the capacity of the energy output of the energy store can be appropriately selected such that the solid matter is not overly accelerated and/or an inner wall of the cannula and the solid matter should be matched to one another such that there is sufficient friction for preventing solid matter leaving the cannula as a result of said friction.

Alternatively, the first energy store can also be dispensed with. In this case the primary plunger is actuated by hand.

The first energy store preferably comprises a first spring, in particular for displacing the primary plunger in the distal direction, wherein the first spring is more particularly embodied as a helical spring. The advantage of using a spring as first energy store is that the required capacity can be optimized by the selection of the type of spring. Furthermore, this also results in the option of selecting a spring with a nonlinear force/distance property. This can be advantageous if static friction has to be overcome at the beginning of the solid matter displacement. Use is preferably made of a helical spring, more particularly a helical compression spring, since these are commercially available in a particularly wide variety and thus can also be obtained in a cost-effective fashion.

Alternatively, provision can also be made for different embodiments of the energy store. By way of example, provision could be made for elastic tensioning means between the primary plunger and the housing, which can for example be embodied as a tension spring, an elastic band or the like. A multiplicity of known alternatives are available to a person skilled in the art, even in respect of the helical spring. By way of example, depending on the geometry, it may be advantageous to provide a conical spring.

The primary plunger is preferably guided in the secondary plunger. This in turn obtains a simple design of the device. To this end, the secondary plunger is preferably designed as a hollow body, more particularly as a hollow cylinder substantially closed on one side. The primary plunger has an elongate rod-shaped section, which can be guided through the syringe body and the cannula, and a contact region at the proximal end, which for example has a disk-like design and can be oriented perpendicularly to a longitudinal axis of the secondary plunger. The secondary plunger can have an axial groove on the inner wall, which groove interacts with a corresponding protruding element of the contact region of the primary plunger in order to prevent rotation of said secondary plunger relative to the primary plunger.

Alternatively, the primary and the secondary plungers can also be guided next to one another in the housing. The contact region need not have a disk-shaped design, but must merely be designed to be suitable for exerting force on the primary plunger by means of the first spring (see below). By way of example, it may be sufficient for the primary plunger to have a hole at a proximal end, through which the distal end of the first spring can be passed, which also results in ensuring the force transmission from the first spring. In this case the primary plunger is preferably protected against twisting with respect to the secondary plunger.

The first spring is preferably arranged in the secondary plunger such that the primary plunger can be axially displaced from the secondary plunger by a spring force from the first spring. The first spring is guided into the cavity of the secondary plunger and the primary plunger is subsequently inserted into the cavity such that the spring is, in a manner of speaking, clamped between the secondary plunger and the contact region of the primary plunger and can be tensioned. Thus the entire spring path required for the displacement of the primary plunger can be housed in the secondary plunger, as a result of which a space-saving design of the device is obtained.

Alternatively, the primary plunger could be inserted together with the secondary plunger, as a result of which the first spring can be dispensed with. To this end the force-transmission mechanism is preferably designed such that it can be deactivated. By way of example, this can be brought about by axial twisting of the secondary plunger, during which the second toothed rack does not engage with the gearwheel. Here the secondary plunger is displaced distally, as a result of which the primary plunger is at the same time displaced into the cannula through the syringe body. The secondary plunger is subsequently withdrawn, whilst the primary plunger is fixed in its position, and axially rotated such that the second toothed rack thereof once again engages with the gearwheel. To this end, provision could furthermore be made for a forced guide by means of grooves and the protruding element. Finally, the primary plunger could also have a longer design such that a plunger head of the primary plunger protrudes out of the secondary plunger in the proximal direction by at least the distance corresponding to the distance through which the solid matter has to be displaced. Thus the primary plunger could at first be manually pushed into the housing in the distal direction and the solid matter could thereby be placed into the cannula and the secondary plunger subsequently could be actuated in order to withdraw the cannula whilst the first plunger is fixed in its position.

The device preferably comprises a second locking device, by means of which the primary plunger can be locked with respect to the secondary plunger, particularly if the first spring is tensioned. The second spring relaxes as a result of deactivating the second locking device and guides the primary plunger out of the secondary plunger. The locking device preferably comprises a radially actuatable resilient element, which is embodied, more particularly integrally, with the secondary plunger. The resilient element comprises a first protruding element, which projects axially inwards and is in contact with a contact region of the primary plunger if a locking device is active. If the first spring is tensioned, the contact region of the primary plunger is proximal with respect to the protruding element and thus interacts with the latter. If the spring is relaxed, the protruding element is proximal, behind the contact region of the primary plunger.

In a further preferred embodiment, the primary plunger is connected to axially and proximally oriented latching elements, which can be latched into an opening arranged distally in the secondary plunger and can be unlocked by the actuation element. This provides a particularly simple second locking device. The latching elements can be designed such that they can pivot radially, wherein radial pivoting can be brought about by the actuation element.

A person skilled in the art is aware of a multiplicity of further suitable locking devices, which are also suitable in the present case. By way of example, the first protruding element could also interact with the spring rather than with the contact region of the plunger. If the primary plunger is actuated by hand, the second locking device can also be dispensed with.

The secondary plunger preferably comprises an actuation element, by means of which the second locking device can be unlocked manually. Here the actuation element is designed such that it interacts with the resilient element, more particularly that it can drive the first protruding element radially outward.

The actuation element is preferably arranged on a proximal end of the secondary plunger and more particularly is embodied as a pushbutton. To this end, the second locking element preferably has a resilient element with a second protruding element, which has a beveled region with respect to an axial direction. The second protruding element is preferably arranged proximally with respect to the contact region of the primary plunger when the spring is tensioned. The second locking device can be unlocked by means of a pin, which can be displaced axially in the direction of the beveled region and more particularly is connected to the actuation element. When the pin is displaced in the distal direction, it contacts the beveled region of the second protruding element of the resilient element and drives the latter radially outwards. The first spring subsequently relaxes, and guides the primary plunger through the syringe body into the cannula. The pin is preferably connected to a cylinder closed on one end, which can be guided over the proximal end of the secondary plunger such that it fits and is connected to the base in the cylinder, preferably at right angles, wherein the secondary plunger has a corresponding opening, through which the pin can be passed through.

Alternatively, the second locking device could also be activated by action at a distance as a result of the cannula being inserted into the body. To this end, an axially displaceable element could be provided at the distal end of the housing, more particularly on the housing base, which element can interact with the second locking device. Furthermore, the actuation element can also be provided laterally on the secondary plunger. To this end, the resilient element can for example be embodied like a rod and connected centrally to the secondary plunger such that a pivoting movement can be achieved by exerting pressure thereon, as a result of which the first protruding element can be guided radially outward. A person skilled in the art is aware of course of further suitable embodiments of an actuation element.

The device preferably comprises a sleeve, which is mounted on the distal end of the housing in an axially displaceable fashion and, in a first state, is substantially arranged within the housing and, in a second state, at least in part protrudes from the housing. The distal end of the primary plunger is typically contaminated after the injection, i.e. tissue remains or bodily fluids such as blood can adhere to the primary plunger. As a result of designing a displaceable sleeve at the distal end of the housing, the end of the primary plunger protruding from the housing can now be covered and so the user, the human or animal to be injected and third parties do not come into contact with the contaminated region, and more particularly cannot be infected by pathogens or the like. Furthermore, this can also prevent objects or depositing surfaces from becoming contaminated. After the injection the sleeve is preferably present in the second state. However, it may also be in the second state until just before the injection, and so the cannula is protected and undesired injuries are also prevented thereby. After the solid matter was injected, it is merely the primary plunger which protrudes beyond the distal end of the housing.

Alternatively, the sleeve can also be mounted on the outside of the housing such that it can be displaced axially.

The sleeve is preferably designed as a cylinder substantially closed on one side, more particularly as a hollow cylinder. There is an opening in the base of the cylinder, which opening is large enough that the cannula can be passed through. The sleeve moreover has lateral incisions oriented in the axial direction, by means of which the sleeve is guided in the housing such that it can be displaced axially.

Instead of the sleeve, provision can also be made for a protective cap that can be placed over the distal end of the housing and thus protects the cannula, or the user and third parties. The protective cap is removed before the device is used and can be replaced after use. The displaceable sleeve can alternatively also be dispensed with.

The device preferably comprises a second energy store, wherein energy from the second energy store can be used for the distal displacement of the sleeve. The second energy store is preferably activated before the primary plunger is pulled out of the body, and so the sleeve is pushed against the body whilst the primary plunger is being pulled from the body. This already prevents contamination during the pulling out of the primary plunger.

Alternatively, the sleeve can also be designed such that it can be actuated by hand. To this end, the sleeve can also be mounted in an axially displaceable fashion outside of the housing such that the sleeve can be grasped and displaced. Provision can furthermore be made for latching, by means of which the sleeve can be latched in the first state and the second state.

In one state, more particularly after the device has been used, the cannula is preferably situated entirely within the housing, with the primary plunger protruding distally and the sleeve partly protruding from the housing and with the sleeve completely covering a region of the primary plunger that protrudes from the housing. As a result, both the user and the human or animal to be injected are protected from injury by the cannula tip and from contamination.

A second energy store preferably comprises a second spring, which can act between the housing and the sleeve and is tensioned, particularly in the first state. The second spring is preferably tensioned until the syringe body and/or the cannula are completely withdrawn. The distal end of the primary plunger is typically still in the body while the second spring relaxes. The second spring then at first presses the sleeve against the body. It is only when the device, more particularly the primary plunger, is removed from the body that enough space is made between the housing and the body such that the sleeve can be guided out of the housing with the aid of the second spring. Thus contaminated regions of the device are always screened thereby while the primary plunger is being removed from the body, resulting in a safe injection of solid matter being possible. Compared to a sleeve actuatable by hand in particular, the user thereby need not handle the region of the cannula or the contaminated end of the primary sleeve.

Provision can alternatively also be made for a different second energy store. By way of example, it can be implemented by means of a stretchable elastic element such as an elastic band. Finally, the second energy store can also be dispensed with, with the sleeve being actuated by hand. However, in this case the user must be aware that they could be contaminated by the region of the distal end of the device after it has been used.

The second spring is preferably designed as a helical spring. The advantage of a helical spring is that it can be installed into the device in a simple and cost-effective manner.

Alternatively, use can also be made of other springs known to a person skilled in the art, e.g. conical springs; more particularly the shape of the spring can also be matched to the housing or the sleeve.

The device preferably comprises a third locking device, by means of which the sleeve can be locked in the housing in respect of an axial displaceability. To this end, the sleeve comprises radially protruding resilient elements on a proximal outer region, which elements can latch into corresponding recesses in the housing. In order to implement the resilience of the elements, the sleeve can be embodied with an at least slightly resilient material, as a result of which the resilient elements can be designed integrally with the sleeve. This makes cost-effective production possible.

Alternatively, the sleeve can also be advanced so far in the basic state by means of the spring that the cannula is completely covered by the sleeve and is only withdrawn proximally when a force is exerted, more particularly when it is pressed against the body, as a result of which the third locking device can be dispensed with. However, this makes the insertion of the cannula, more particular an insertion of the cannula that is not perpendicular to the body surface, more difficult. The puncturing site cannot be precisely localized through the sleeve. Furthermore, in the case of a non-perpendicular insertion of the cannula, a radial force is exerted on the sleeve, as a result of which the latter can twist.

The third locking device can preferably be unlocked in the first state and cannot be unlocked in the second state in particular. The third locking device can preferably be unlocked in the first state and so the sleeve can be displaced distally by the second energy store, more particularly by the second spring, such that the primary plunger and/or the cannula are covered. If the sleeve is only guided to the front after the injection has been completed, it is advantageous if the sleeve in the advanced state is locked irreversibly by means of the third locking device, in particular such that it cannot be unlocked by simple means. This reduces the risk of contamination because inappropriate handling cannot inadvertently withdraw the sleeve and uncover the contaminated region. Depending on the design of the sleeve, this can also prevent a reuse of the device. To this end, the sleeve can be designed to be sufficiently long such that, after the secondary plunger has been withdrawn, the cannula cannot protrude from the sleeve. It would also be feasible to provide the opening in the sleeve with a resilient flap like a nonreturn valve, which flap is kept open against the spring force during use by means of the cannula or plunger and closes after the sleeve has been extended over the primary plunger.

Alternatively, particularly if the device is provided for multiple uses, the third locking device can also be designed such that it cannot be locked in the second state. If the sleeve is in the second state as standard, the third locking device can also only be lockable and unlockable in the second state. Finally, the third locking device can also be dispensed with completely.

The third locking device is preferably designed such that it can be deactivated by the secondary plunger. This is advantageous in that the user does not have to actively or consciously deactivate the latter and thus cannot forget to deactivate it either. The third locking device is preferably deactivated when the second plunger has been completely inserted, particularly because in this state the cannula is completely withdrawn and the injection has, so to speak, been completed. If no great force acts on the sleeve from the second energy store, the third locking device can also already have been deactivated before this. However, the third locking device is preferably deactivated no sooner than the introduction of the cannula into the body.

The sleeve comprises at least one outwardly directed element in a proximal region, which element can however also be provided such that it encircles the sleeve. A distal region of the element has a flank, which is at right angles on the sleeve and thus can interact with corresponding recesses or radially inwardly directed elements of the housing. A proximal region of the element has a beveled flank, which tends radially outwards in the distal direction. The secondary plunger is designed such that it can interact with the beveled flank and can drive the outwardly directed element of the sleeve radially inwards, more particularly out of the groove or from the engagement with the element of the housing directed radially inwards. The sleeve can subsequently be displaced in the distal direction by means of the second spring.

If the deactivation should take place directly after the insertion of the cannula into the body, provision can be made for an actuation element, for example in a distal region of the housing of the device, more particularly in a contact region of the housing with the body, or on the sleeve itself.

The syringe body preferably comprises a retention device for retaining the solid matter before the injection. The device is typically exposed to tremors during the transportation or before the cannula is inserted into the body, as a result of which the solid matter can slip out of the syringe body. By forming a retention device, the solid matter now remains in the syringe body before the device is used, more particularly before the primary plunger is displaced. To this end, the syringe body can for example comprise inwardly projecting, resilient lugs. Furthermore, the retention device can be designed as at least one elongate section, which is resiliently connected to the syringe body in the axial direction via a proximal end, more particularly, it is integrally connected or by means of a clip-on connection and can have lugs projecting inwardly into the interior of the syringe body in a proximal and in a distal region. The solid matter can either be arranged between the lugs or be clamped by one or more lugs. The retention device is deactivated by interaction of the primary plunger with the proximally arranged lug, as a result of which the distal lug is guided radially outwards over the elongate section. A further lug spaced in the proximal direction from the lugs holding the solid matter can also be provided for deactivating the retention device, which further lug is driven radially outwards by means of the primary plunger.

Alternatively, the syringe body may merely have a cross-sectional restriction, and so the solid matter is held here coupled by friction. The cross-sectional restriction can also be implemented by means of elements that are resilient in the radial direction. Finally, the retention device could also be dispensed with. In this case it is advantageous if the device comprises a cap for the cannula, which cap can prevent premature emergence of the solid matter. By way of example, this can be implemented by a simple cap or a pin inserted into the cannula. However, care has to be taken in this case that the cannula is not directed downwards after the cap has been removed because otherwise the solid matter could thereby fall out of the device.

The device preferably comprises a tongue, more particularly a tongue made of sharp-edged metal connected to the housing and engaging with the syringe body. This can prevent the syringe body from being able to be displaced in the distal direction. The advantage of this is that the cannula also can thereby not be displaced back into the body while it is being withdrawn therefrom. Furthermore, the embodiment of a tongue is advantageous in that this can bring about a latch-free distal withdrawal prevention. This is particularly advantageous because the user of the device can insert the secondary plunger in a continuous fashion. The embodiment of this device for blocking a movement direction can be designed similar to that of a cable strap. In a further preferred embodiment, the device comprises a tongue, which is pivoted radially outwards in the distal direction and engages with the toothed rack of the secondary plunger, as a result of which a proximal displacement of the secondary plunger can be prevented. In this case the tongue can consist of both metal and plastics or other materials, which are preferably at least slightly elastic. The noise caused thereby imparts the feeling of solid functioning of the device to the user. This more particularly brings about a latching withdrawal prevention with a particularly simple design.

The tongue can alternatively also interact with the secondary plunger. The tongue can also either be connected to the secondary plunger and interact with the syringe body or vice versa. Furthermore, the tongue can also interact with the toothed racks of the syringe body, wherein the tongue would in this case be arranged pivoted radially inwards in the proximal direction in order to block a corresponding movement of the syringe body in the proximal direction. Finally, the gearwheel can also be designed such that it can only be rotated in one direction, wherein the gearwheel can for example be designed as a ratchet wheel, known from mechanical clockworks.

The cannula, the first and the second springs preferably consist of steel, but use can also be made of plastics. By way of example, polymethyl methacrylate (PMMA), polycarbonate or other plastics known to a person skilled in the art can be used for the remaining parts of the device, wherein, more particularly, different plastics can also be used for different parts.

Arbitrary dimensions may be selected for the device and may, in particular, be matched to the solid matter. The penetration depth can be regulated by selection of the cannula length.

In the following text, a possible procedure for injecting solid matter with an embodiment of the device according to the invention is described, step by step:
1. In a first step, a protective cap, if present, is removed from the cannula and the cannula is subsequently inserted into the body of the human or animal to be injected.
2. Once the cannula has been inserted, the actuation element in the proximal region of the secondary plunger is activated, as a result of which
   a. the second locking device is unlocked; and, as a result of which
   b. the first spring is relaxed; leading to
   c. the primary plunger transferring solid matter from the syringe body into the cannula as a result of the spring force of the first spring; whereupon
   d. the primary plunger comes to rest at least in part in the cannula and the solid matter comes to rest completely outside of the housing; and
   e. the first locking device is deactivated.
3. The secondary plunger is subsequently displaced in the distal direction; as a result of which
   a. the syringe body and/or the cannula is/are displaced in the proximal direction whilst the primary plunger remains fixed in its position, and so the cannula is, more particularly completely, withdrawn into the syringe body; and
   b. the third locking device is unlocked; as a result of which
   c. the second spring relaxes and the sleeve is in part displaced out of the housing in the distal direction and more particularly comes into contact with the body.
4. Finally the syringe is removed from the body, wherein, more particularly, it is only the secondary plunger that is pulled out of the body. At the same time this pushes the sleeve completely over the part of the plunger protruding from the housing by means of the second spring, whereupon the sleeve latches.

The device comprises no sleeve in a further preferred embodiment. In order nevertheless to be able to ensure safe use, more particularly safe handling after the device has been used, the device can be configured as described below.

In one state, more particularly after the device has been used, the cannula and a distal end of the primary plunger are preferably situated entirely within the housing. This ensures a particularly safe use of the device because contamination after use can largely be avoided thereby. In particular, this provides an alternative embodiment to the above-described embodiment with the sleeve that surrounds the distal end of the plunger after use. The cannula and the primary plunger are preferably also withdrawn by means of the force-transmission mechanism.

The withdrawal of the cannula and the primary plunger can, in variants, also be dispensed with, particularly if, for example, provision is made for an above-described sleeve.

In a second state, in which the secondary plunger is partly inserted, the cannula is preferably withdrawn proximally, and so the solid matter is not held in the cannula and the primary plunger is at best partly held by the cannula, or, in a third state, when the secondary plunger is completely inserted, more particularly after the device has been used, the primary plunger and the cannula are withdrawn together with respect to the first state. The plunger and the cannula are preferably withdrawn in sequence. The solid matter is placed into the cannula in a first state. In a next step, the cannula is withdrawn via the force-transmission mechanism by distal insertion of the secondary plunger, whilst the primary plunger remains fixed in its position, as a result of which the second state is reached. Subsequently the cannula and the primary plunger are withdrawn simultaneously by continued insertion of the secondary plunger until finally, in a third state, the distal end of the primary plunger and the cannula are completely situated within the housing.

The cannula can, in variants, be completely withdrawn into the housing directly after the solid matter was placed into the cannula.

The primary plunger preferably comprises a plunger head at a proximal end, wherein the plunger head is distanced from the syringe body in a first state, when the spring is relaxed before the secondary plunger is inserted, and the syringe body contacts the plunger head in the second state. Thus a particularly simple sequential withdrawal of the cannula or primary plunger is obtained. After the first spring has relaxed, the primary plunger is prevented from further displacement in the distal direction by a stop. This is advantageous because this means that the first spring does not have to relax completely, as a result of which a reproducible end position of the primary plunger can be ensured in any position of the device (horizontal, vertically upwards or downwards, etc.). By way of example, the stop can be embodied by the first locking device. In this state, that is to say before the secondary plunger is inserted, the plunger head is now distanced from the syringe body. If the secondary plunger is now inserted, it firstly glides along the primary plunger. As soon as the syringe body contacts the plunger head, the cannula is withdrawn to the extent that it no longer surrounds the solid matter. The distal end of the primary plunger is preferably surrounded substantially flush by the cannula in this state, wherein the primary plunger can also protrude distally over the cannula. If the secondary plunger is now inserted further, the syringe body pushes back the primary plunger in the proximal direction via the plunger head, that is to say the cannula and the primary plunger are displaced together in the proximal direction. When the secondary plunger is completely inserted, the cannula and the distal end of the primary plunger are completely situated within the housing. In this state the device can now be handled with substantially no risk.

The sequential withdrawal can also be implemented differently in variants. The primary plunger could also comprise a lateral stop for the syringe body. A person skilled in the art is also aware of further options to this end.

The primary plunger preferably comprises a first, proximal section and a second, distal section, wherein the first section has a larger diameter than the second section. This also allows solid matter with very small dimensions, more particularly diameters, to be administered without endangering the stability of the device. The second section with the smaller diameter can be guided in sections such that the functionality of the device is not adversely affected by a bend in the second section as a result of gravity or other accelerations (fast pivot movements by the user of the like). If the second region has a sufficiently stiff design, for example if it is made of a non-elastic material, the guiding can also be dispensed with. With respect to the retention device in particular, which comprises an elongate element with lugs protruding into the interior of the syringe body, it may be advantageous if the region, which contacts the lugs that should be driven radially outwards, has a sufficiently stable design. In this case, the elongate element preferably comprises three lugs, with two distally arranged lugs holding the solid matter and the third lug, spaced apart in the proximal direction, interacts with the first, more stable section of the primary plunger. Hence, it is sufficient for the syringe body to have an inner diameter corresponding to the outer diameter of the second section only in the medicament chamber and in the cannula. Proximally behind the two lugs that hold the solid matter, the syringe body may have an inner diameter corresponding to the outer diameter of the first section.

In variants, the above-described embodiment of the primary plunger may also be dispensed with, particularly if, for example, the solid matter has sufficiently large dimensions such that the stability and the functionality of the retention device can be ensured.

The syringe body preferably comprises a radial bore in the region of the solid matter uptake. This provides a solid-matter-sparing fixing option for the solid matter whilst the device is being loaded with solid matter. The solid matter can be held in position after placement by a vacuum by means of the radial bore, until the solid matter is held by the lugs in the elongate section of the retention device. Furthermore, this provides the option of checking for the presence of the solid matter during or after the production of the device.

The radial bore can also be dispensed with in variants. Checking for the presence of the solid matter could for example be made possible by a syringe body with a transparent design or by a window in the syringe body.

Further advantageous embodiments and combinations of features of the invention emerge from the following detailed description and the entirety of the patent claims.

### BRIEF DESCRIPTION OF THE DRAWINGS

In the drawings used to explain the exemplary embodiment:

FIG. 1 shows a schematic illustration of a cut along the longitudinal axis of a first embodiment of the device according to the invention;

FIG. 2 shows an illustration as per FIG. 1 after the cannula was inserted into the body;

FIG. 3 shows an illustration as per FIG. 2 after the actuation element was actuated;

FIG. 4 shows an illustration as per FIG. 3 after insertion of the secondary plunger;

FIG. 5 shows an illustration as per FIG. 4 after the injection has been completed;

FIG. 6 shows a schematic illustration of a cut along the longitudinal axis of a second embodiment of the device according to the invention after the cannula was inserted into the body;

FIG. 7 shows an illustration as per FIG. 6 after the actuation element was actuated;

FIG. 8 shows an illustration as per FIG. 7 after insertion of the secondary plunger; and

FIG. 9 shows an illustration as per FIG. 8 after the injection has been completed.

In the figures, identical parts have generally been provided with the same reference sign.

### WAYS OF IMPLEMENTING THE INVENTION

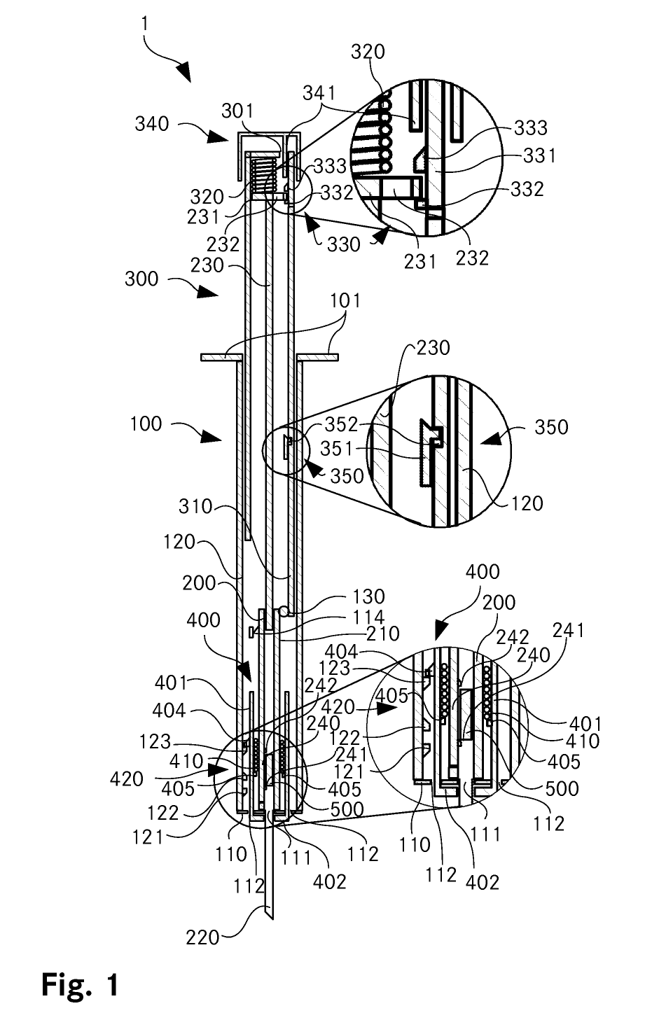

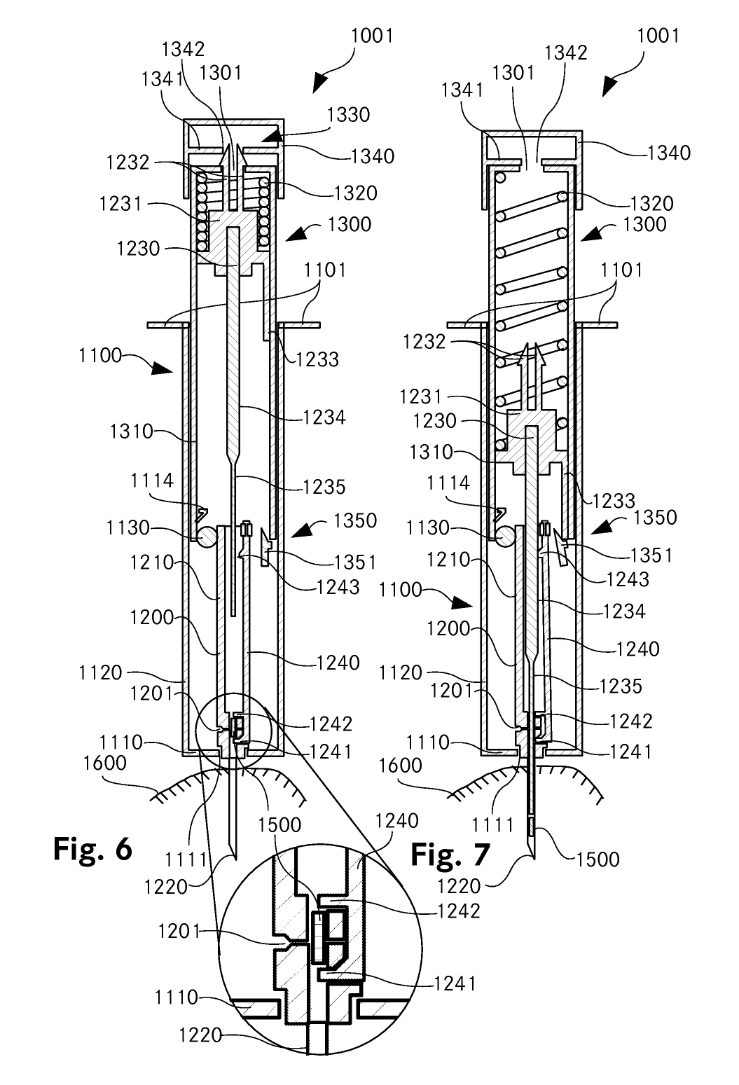

FIGS. 1 to 5 show a first embodiment and FIGS. 6 to 9 show a second embodiment of a device according to the invention. In the following paragraphs, the first embodiment shall be discussed first.

FIG. 1 shows a schematic illustration of a cut along the longitudinal axis of a first embodiment of the device 1 according to the invention, prior to use.

The device 1 comprises a housing 100, which is substantially designed as a hollow cylinder, closed on one side, with the opening oriented in the proximal direction. The device 1 furthermore comprises a primary plunger 230 and a syringe body 200, in which solid matter 500 is held by means of a retention device, in particular before the device 1 is used. The syringe body 200 is fixedly connected to a cannula 220, with the primary plunger 230 being able to be axially displaced through the syringe body 200 and into the cannula 220. The syringe body 200 and the cannula 220 are mounted together in the housing 100 in an axially displaceable fashion. Furthermore, the device 1 comprises a secondary plunger 300, which is likewise mounted in the housing 100 in an axially displaceable fashion and is substantially embodied as a hollow cylinder, closed on one side, with an opening oriented in the distal direction. The primary plunger 230 is mounted in the secondary plunger 300 such that it can be displaced in the axial direction. Finally, the device 1 comprises a sleeve 400, which is mounted in an axially displaceable fashion in a distal region of the housing 100. The sleeve 400 is also designed as a hollow cylinder, substantially closed on one side, with the opening being oriented in the proximal direction.

In the region of the opening, the housing 100 comprises two holding elements 101, which protrude outwards at right angles with respect to a longitudinal axis of the device 1 and, more particularly, can be grasped by means of the index and middle fingers.

On a housing base 110 of the housing 100, the latter comprises a central, circular cannula opening 111, through which a cannula 220 can be passed. Furthermore, a plurality of arc-shaped slits are embodied as sleeve openings 112 on the housing base 110 and surround the cannula opening 111, by means of which slits respectively one sleeve cladding segment of a sleeve cladding 401 of the sleeve 400 is held in an axially displaceable fashion.

On the inside of the wall, a housing cladding 120 of the housing 100 in the region of the housing base 110 encompasses three spaced apart protruding elements 121-123, which are part of a third locking device 420 (see below) and are oriented one behind the other in a row in the axial direction. These protruding elements 121-123 have a wedge-shaped design, with a beveled flank of protruding element 121 closest to the housing base 110 having a distal orientation. The adjacent wedge-shaped protruding element 122 is mirrored with respect to the first element 121 in a plane perpendicular to the longitudinal axis of the housing, and the last wedge-shaped protruding element 123 furthest from the housing base 110 is in turn oriented like the protruding element 121. In the cross section perpendicular to the longitudinal axis of the housing, the protruding elements 121-123 have a cam-shaped design.

Furthermore, the housing 100 comprises a gearwheel 130, which, measured in the axial direction in respect of a length of the housing 100, is mounted approximately centrally and, in respect of a cross-sectional plane of the housing 100, is mounted, offset from the center by approximately half a syringe body diameter of the syringe body 200 in a direction parallel towards the outside, such that it can rotate on the housing 100.

Furthermore, a tongue is connected to the housing 100, distally with respect to the gearwheel 130 and therebelow in the axial direction, which tongue is slightly offset radially inwards with respect to the gearwheel 130 and, in the distal direction, includes an angle of approximately 45° with the syringe body 200. The tongue 114 is preferably formed from sharp-edged metal, more particularly steel, and interacts with the syringe body 200 by means of an at least slightly resilient region such that the syringe body 200 can be displaced in the proximal direction, but a displacement of the syringe body 200 in the distal direction is prevented. The tongue 114 preferably interacts with a smooth region (not with the region embodied as a toothed rack) of the syringe body 200, and so the secondary plunger 300 can be inserted in a continuous fashion.

Furthermore, a locking element 351, as part of a first locking device 350, is connected to the housing 100, which locking element is designed such that it can be pivoted in a substantially radial fashion against a spring force. The locking element 351 is embodied as an axially oriented elongate section and, in the present embodiment, is integrally embodied with the housing 100. The locking element 351 is fixedly connected to the housing 100 in a distal region and, in a proximal region, it has a radially protruding element and, at the proximal end, a beveled flank, which includes an angle of approximately 45° with the primary plunger 230 in the distal direction.

On its lateral surface, the syringe body 200 comprises a first toothed rack 210 (not illustrated explicitly), which is oriented in the axial direction and which, in a first state, engages with the gearwheel 130 over a proximal end. Furthermore, it comprises a retention device, known from EP 09 405 186.9 (Forteq Nidau AG), for retaining the solid matter 500. To this end, the syringe body 200 has an elongate element 240, which is proximally connected to the syringe body 200 in a resilient fashion and which, as a U-shaped recess, is integrally embodied with the syringe body. The elongate element 240 has lugs 241, 242 that protrude radially into the interior of the syringe body 200, wherein one lug 241 is arranged distally and the other lug 242 is arranged in the proximal direction, spaced apart by at least a length of solid matter 500, and so the solid matter 500 can be held between the lugs 241, 242. In order to deactivate the retention device, the proximally arranged lug 242 is driven radially outwards by means of the primary plunger 230, whereupon the distally arranged lug 241 is at the same time driven radially outwards via the elongate section 240 and thus the interior of the syringe body 200 is freed for the solid matter 500.

The secondary plunger 300 is substantially designed as a cylinder closed on one side, with the cylinder opening being distally arranged and the distal region furthermore having a cylinder-cladding-segment-shaped recess. At its proximal, substantially closed end, the secondary plunger 300 has a pin opening 301 and, laterally in the region of the proximal end, a second locking device 330, which comprises a resilient element 331. In the present embodiment, the resilient element 331 is designed as a U-shaped recess in the secondary plunger 300 and connected proximally thereto and has two inwardly protruding elements 332, 333, which are axially spaced apart; with the proximally arranged element 333 having a wedge-shaped design and a beveled flank in the proximal direction. In the cross section that is perpendicular to the longitudinal axis of the housing, the elements 332, 333 have a cam-shaped design. The resilient element 331 is designed such that it can bend radially outwards.

The secondary plunger 300 furthermore comprises an actuation element 340, which is substantially designed as a cylinder closed on one side and which, parallel to the cylinder shell, has a pin 341 connected to the cylinder base. The actuation element 340 is placed over the proximal end of the secondary plunger 300, with the pin 341 being guided through the pin opening 301 and, in a first state, being arranged axially in the proximal direction in respect of the element 333; more particularly, the actuation element 340 is not completely inserted in the first state. While the holding elements 101 are grasped by the index and middle fingers, the actuation element 340 is typically actuated by the thumb, namely by moving the thumb in the direction of the index and middle fingers and thus displacing the secondary plunger 300 in the distal direction.

From the distal region, the secondary plunger 300 is equipped with an axially oriented second toothed rack 310 on the inner wall, which toothed rack is more particularly integrally embodied with the secondary plunger 300 and can interact with the gearwheel 130 of the housing 100. In a first state, a distal region of the second toothed rack 310 engages with the gearwheel 130. Hence the movement of the secondary plunger 300 is coupled to that of the syringe body 200, wherein a distal movement of the secondary plunger 300 is converted into a proximal movement of the syringe body 200 via the rack-and-pinion gear formed thereby, and with an opposite movement being prevented by the tongue 114.

The primary plunger 230 guided in the secondary plunger 300 has a disk-shaped plunger head 231 with an opening 232 at its proximal end. The diameter of the plunger head 231 substantially corresponds to the internal diameter of the secondary plunger 300. The plunger head 231 and the secondary plunger 300 enclose a first spring 320, which, in a first state, is tensioned. This first spring 320 is designed as a helical spring and consists of steel. The plunger head 231 is held between the elements 332, 333 of the secondary plunger 300; more particularly, a force is exerted on the element 332 in the distal direction as a result of the tensioned first spring 320.

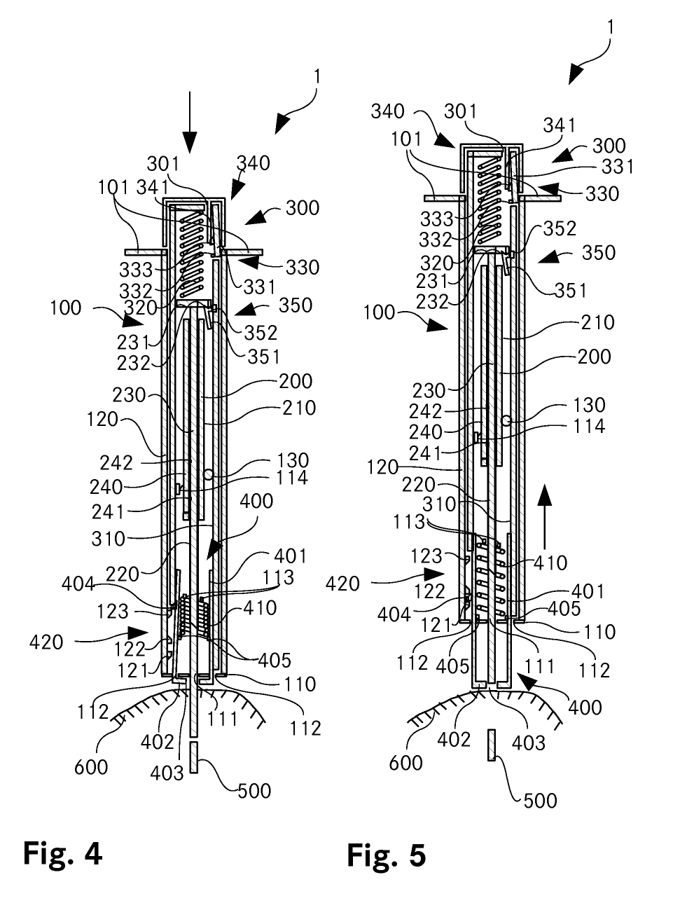

The sleeve 400, which is mounted in the housing such that it can be axially displaced and embodied as a hollow cylinder substantially closed on one side, comprises, on a sleeve base 402 of the sleeve 400, a cannula opening 403 for the cannula 220. The sleeve cladding 401 has incisions (not visible), which are designed corresponding to the sleeve opening 112 of the housing 100, and is guided in the sleeve opening 112 in the housing in an axially displaceable fashion. The housing 100 has radially inwardly protruding retention elements 113 (visible in FIGS. 4 and 5), which protrude into the interior of the sleeve 400 through the incisions in the sleeve cladding 401. In a first state, the inner side of the sleeve base 402 contacts the outer side of the housing base 101. A second spring 410, tensioned in the first state, is held by the retention elements 113 in the housing 100 in a proximal region and held by retention elements 405 in the sleeve 400 in a distal region. The second spring 410 is designed as a helical spring and consists of steel. On the outer side, the sleeve cladding 401 is connected to a wedge-shaped protruding element 404 with a square-shaped attachment, with the beveled flank of the protruding element 404 being oriented in the proximal direction.

In the subsequent paragraphs, the use of the device 1 for injecting solid matter is now explained in more detail with the aid of FIGS. 1 to 5.

Before use, the device 1 is in the state as per FIG. 1.

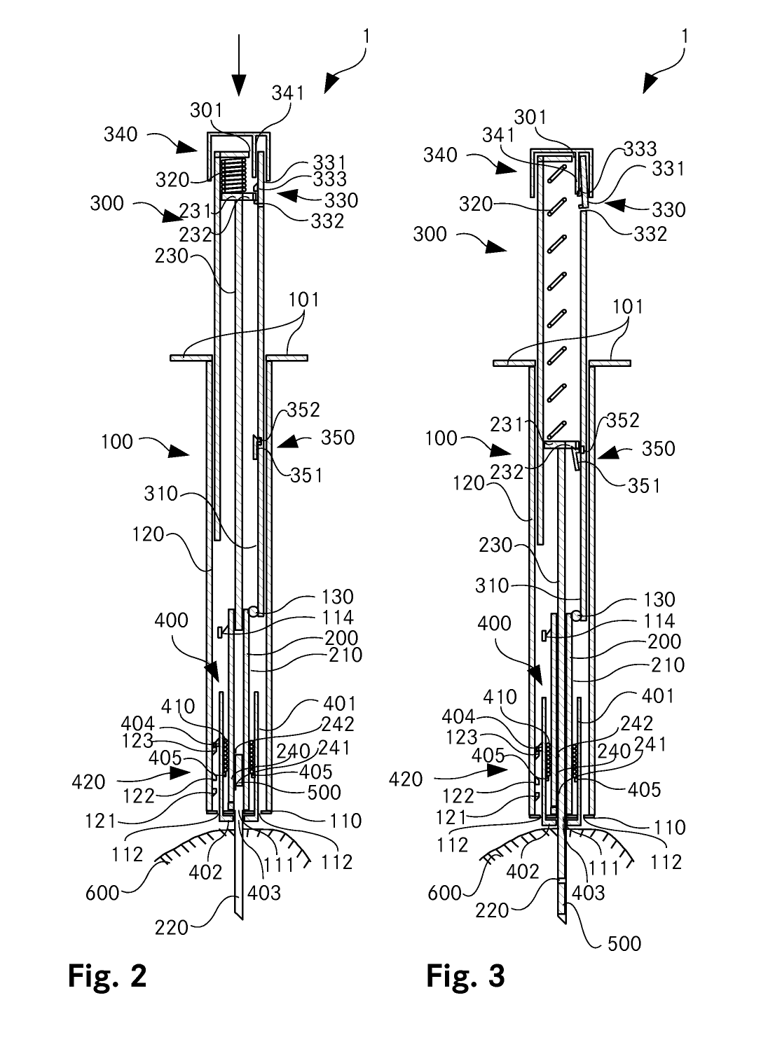

FIG. 2 shows a schematic illustration of an embodiment of the device 1 according to the invention as per FIG. 1 after the cannula 220 was inserted into a body 600, more particularly as a first step when injecting solid matter, before the actuation element 340 is actuated.

The device 1 is now grasped by the user on the holding elements 101 by means of the index and middle finger, and the thumb is placed, initially without pressure, onto the actuation element 340.

FIG. 3 shows a schematic illustration as per FIG. 2 after the actuation element 340 was actuated. If the actuation element 340 is now actuated in the distal direction by means of the thumb, the pin 341 is thereby displaced in the distal direction and thereupon contacts the flank of the element 333, thus bending the locking element 331 radially outwards. The element 332, which is coupled thereto, is also driven radially outwards, as a result of which the element 332 no longer engages with the plunger head 231 of the primary plunger 230. Thus the path of the primary plunger 230 is freed in the distal direction. The first spring 320 subsequently relaxes, as a result of which the primary plunger 230 is displaced relative to the secondary plunger 300 in the distal direction.

In the syringe body 200, the distal end of the primary plunger 230 first of all reaches the first lug 242 and drives the latter radially outwards. Coupled to this, the elongate section 240, and hence the lug 241 as well, are driven radially outwards, as a result of which the path for the solid matter 500 is freed. Then, driven by the still partly tensioned first spring 320, the primary plunger 230 captures the solid matter 500 with its distal end and guides said solid matter into the cannula 220, more particularly into a region of the cannula 220 situated outside of the housing 100. The spacing between the solid matter and the housing 100, or the cannula opening 403 of the sleeve 400, substantially corresponds to an envisaged implant depth of the solid matter 500 in the body 600.

Thus, in this state, a substantial region of the cannula 220 is in the body 600, a distal region of the primary plunger 230 is in the cannula 220, and hence also in the body 600, and the solid matter 500 is within the body 600 in front of the distal end of the primary plunger 230.

Concurrent with the complete insertion of the primary plunger 230, the plunger head 231 contacts the locking element 351 of the first locking device 350. More particularly, the opening 232 of the plunger head 231 drives the beveled flank of the locking element 351 radially inwards, as a result of which a protruding element of the locking element 351, which former element protrudes radially outwards and was previously situated in a recess 352 of the secondary plunger, is lifted radially inwards and out of said recess 352. Hence the first locking device 350 is unlocked, and so the secondary plunger 300 can be displaced axially within the housing 100. The secondary plunger 300 is in turn inserted by means of pressure on the actuation element 340 exerted by the thumb.

If the secondary plunger 300 is now inserted into the housing 100 in the distal direction by pressure exerted on the actuation element 340, a proximal movement of the syringe body 200, and hence the cannula 220 as well, is brought about at the same time via the first and the second toothed rack 210, 310, which are coupled via the gearwheel 130; at the same time the primary plunger 230 remains fixed in its position. As a result, the cannula 220 is withdrawn back over the solid matter 500 and the region of the primary plunger 230 protruding out of the housing 100, and so the solid matter 500 remains outside and finally only a region of the primary plunger 230 protrudes out of the housing.

FIG. 4 shows a schematic illustration as per FIG. 3 after the insertion of the secondary plunger 300. When the secondary plunger 300 is inserted by actuating the actuation element 340, the first spring 320 is recompressed. The solid matter 500 and a distal region of the primary plunger 230 remain in the body 600, while the cannula 220 is withdrawn over the solid matter 500 and over the distal region of the primary plunger 230. Meanwhile the solid matter 500 is retained in the body 600 by the primary plunger 230, which is fixed in its position.

So that the secondary plunger 300 cannot be displaced in the proximal direction by the first spring 320 when the former is released, provision is made for the tongue 114, which acts in a resilient fashion on the lateral surface of the syringe body 200, thus twisting together with the syringe body 200 when a proximal force is exerted on the secondary plunger 300 and so prevents the secondary plunger 300 from returning into the body 600, or prevents distal displacement of the cannula 220 back into said body.

When the secondary plunger 300 is inserted completely, it contacts the protruding element 404 of the sleeve 400 with the region of the cylinder-cladding-segment-shaped recess, by means of which the protruding element 404 is driven radially inwards and thereby lifted over the protruding element 123 of the housing 100. This unlocks the third locking device 420, whereupon the second spring 410 can relax and thus the sleeve 400 can be extended out of the housing 100, more particularly over the region of the primary plunger 230 protruding out of the housing 100. However, in this state, the region of the primary plunger 230 protruding out of the housing 100 is still in the body 600, as a result of which the sleeve 400 is at a first time pressed against the body 600 by means of the second spring 410 and thus cannot be completely extended.

FIG. 5 shows a schematic illustration as per FIG. 4 after the injection has been carried out. After the third unlocking device 420 has been unlocked, the device is carefully pulled out of the body 600, more particularly the distal region of the primary plunger 230 is pulled out of the body 600, while the solid matter 500 remains therein. At the same time, the second spring 410 can now relax completely, and thus the sleeve 400 can be guided out of the housing 100 and over the primary plunger 230. In the meantime, the protruding element 404 of the sleeve 400 reaches the next protruding element 122 of the housing 100, is lifted over said protruding element 122 by the spring force as a result of the wedge-shaped design of said element and latches between the protruding elements 122 and 121, more particularly in an irreversible fashion. In this state, the region of the primary plunger 230 protruding out of the housing 100 is completely covered by the sleeve 400, and this can prevent possible contaminations.

The cannula 220, the first spring 320 and the second spring 410 consist of metal, more particularly of steel. The remaining parts of the device 1 are made of a plastic, for example, polymethyl methacrylate (PMMA) or polycarbonate. A person skilled in the art also knows of further suitable materials that can be used for individual parts of the device 1. By way of example, the first and the second spring 320, 410 could also be made of a plastic.

The holding elements 101 can also be designed as a circular disk encircling the housing or as further holding elements 101 known to a person skilled in the art. In particular, provision can also be made for an ergonomically adapted shape of the holding elements 101.

The protruding elements 121-123 and/or the toothed racks 210, 310 can also have an encircling design. An encircling design of the protruding elements 121-123 or the toothed racks 210, 310 can make the device resistant in respect of rotations of the housing, the secondary plunger, the sleeve and the syringe body with respect to each other.

The tongue 114 can also interact with the gearwheel 130 or with the secondary plunger 300.

FIGS. 6 to 9 show a second embodiment of the device according to the invention

The device 1001, as per the second embodiment, comprises a housing 1100, which is substantially designed as per the first embodiment. The device 1001 furthermore comprises a primary plunger 1230 and a syringe body 1200, in which solid matter 1500 is held by means of a retention device, in particular before the device 1001 is used. The syringe body 1200 is fixedly connected to a cannula 1220, with the primary plunger 230 being able to be axially displaced through the syringe body 1200 and into the cannula 1220. The syringe body 1200 and the cannula 1220 are mounted together in the housing 1100 in an axially displaceable fashion. Furthermore, the device 1 comprises a secondary plunger 1300, which is likewise mounted in the housing 1100 in an axially displaceable fashion and is substantially embodied as a hollow cylinder, closed on one side, with an opening oriented in the distal direction. The primary plunger 1230 is mounted in the secondary plunger 1300 such that it can be displaced in the axial direction.

In the region of the opening, the housing 1100 comprises two holding elements 1101, which protrude outwards at right angles with respect to a longitudinal axis of the device 1001 and, more particularly, can be grasped by means of the index and middle fingers. On a housing base 1110 of the housing 1100, the latter comprises a central, circular cannula opening 1111, through which a cannula 1220 can be passed.

Furthermore, the housing 1100 comprises a gearwheel 1130, which, measured in the axial direction in respect of a length of the housing 1100, is mounted approximately centrally and, in respect of a cross-sectional plane of the housing 1100, is mounted, offset from the center by approximately half a syringe body diameter of the syringe body 1200 in a direction parallel towards the outside, such that it can rotate on the housing 1100.

Furthermore, a tongue 1114 is connected to the housing 1100 proximally behind the gearwheel 1130, which tongue is pivoted radially outwards in the distal direction and engages with the second toothed rack 1310, by means of which a proximal displacement of the secondary plunger 1300 is prevented indirectly. The tongue 1114 consists of an at least slightly elastic plastic.

Furthermore, a locking element 1351, as part of a first locking device 1350, is connected to the housing 1100, which locking element is designed such that it can be pivoted in a substantially radial fashion against a spring force. The locking element 1351 is embodied as an axially oriented elongate section and, in the present embodiment, is integrally embodied with the housing 100. The locking element 1351 is fixedly connected to the housing 1100 in a distal region and, in a proximal region, it has a radially protruding element and, at the proximal end, a beveled flank, which includes an angle of approximately 45° with the primary plunger 230 in the distal direction. The secondary plunger 1300 can thereby be locked in the distal direction.

On its lateral surface, the syringe body 1200 comprises a first toothed rack 1210 (not illustrated explicitly), which is oriented in the axial direction and which, in a first state, engages with the gearwheel 1130 over a proximal end. Furthermore, it comprises a retention device, known from EP 09 405 186.9 (Forteq Nidau AG), for retaining the solid matter 1500. To this end, the syringe body 1200 has an elongate element 1240, which is proximally connected to the syringe body 1200 in a resilient fashion via a clip-on connection. The elongate element 1240 has lugs 1241, 1242 that protrude radially into the interior of the syringe body 1200, wherein one lug 1241 is arranged distally and the other lug 1242 is arranged in the proximal direction, spaced apart by at least a length of solid matter 1500, and so the solid matter 1500 can be held between the lugs 1241, 1242. Furthermore, spaced proximally from the proximal lug 1242, there is a third lug 1243, which protrudes radially inwards and is connected to the elongate element 1240. Furthermore, a radial bore 1201, which protrudes into the interior of the syringe body 1200, is provided in the region between the two distal lugs 1241 and 1242. This can be used to hold the solid matter 1500 in its location by means of a vacuum while the device is equipped, or the presence of the solid matter 1500 can be checked by means of a suitable probe. An internal diameter of the syringe body 1200 in front of the two distally arranged lugs 1241 and 1242 is greater than the internal diameter of the syringe body 1200 between the distally arranged lugs 1241 and 1242 (see below for more details). In terms of magnitude, the cannula 1220 substantially has the same internal diameter and the solid matter 1500 substantially has the same external diameter as the internal diameter of the syringe body 1200 in the region between the distally arranged lugs 1241 and 1242.

In order to deactivate the retention device, the proximally arranged lug 1243 is driven radially outwards by means of the primary plunger 1230, whereupon the distally arranged lugs 1241 and 1242 are at the same time driven radially outward via the elongate section 1240 and thus the interior of the syringe body 1200 is freed for the solid matter 1500.

The primary plunger comprises an elongate region, which has a first section 1234 and a second section 1235, which is arranged distally with respect to the first section, wherein the first section 1234 has a greater diameter than the second section 1235. The transition region between the first section 1234 and the second section 1235 has a conical design, with the flank of the cone serving as a contact region for the lug 1243.

The secondary plunger 1300 is substantially designed as a cylinder closed on one side, with the cylinder opening being distally arranged. At its proximal, substantially closed end, the secondary plunger 1300 has an axially and centrally arranged latching element opening 1301 as part of a second locking device 1330 (see further down).

The secondary plunger 1300 furthermore comprises an actuation element 1340, which is substantially designed as a cylinder, closed on one side, with an intermediate plane 1341. As a further part of the second locking device 1330, the intermediate place 1341 has an unlocking opening 1342, which is arranged coaxially with respect to the latching element opening 1301 and has a similar diameter. The actuation element 1340 is placed over the proximal end of the secondary plunger 1300.

The primary plunger 1230 guided in the secondary plunger 1300 has a plunger head 1231 at its proximal end. The diameter of the plunger head 1231 substantially corresponds to the internal diameter of the secondary plunger 1300 and, centrally and distally, has a bore for holding the first section 1234. Furthermore, the plunger head 1231 comprises two axially and proximally arranged latching elements 1232, which are arranged spaced apart in the vicinity of the longitudinal axis of the plunger head 1231 and have a radially resilient design. The latching elements 1232 have radially outwardly protruding lugs, which have inwardly leaning flanks in the proximal direction. When the second locking device 1330 is locked, the latching elements 1232 protrude through the latching element opening 1301, with the edge region of the latching element opening 1301 distally undercutting the lugs. The plunger head 1231 and the secondary plunger 1300 enclose a first spring 1320, which is tensioned in a first state when the second locking device 1330 is locked. This first spring 1320 is embodied as a helical spring and consists of steel. If the actuation element 1340 is actuated at this point, the latching elements 1232 are pivoted radially inwards through the unlocking opening 1342, as a result of which the lugs are no longer undercut by the latching element opening 1301. This unlocks the second locking device 1330, whereupon the latching elements 1232 are guided through the latching element opening 1301 as a result of the first spring 1320 relaxing. The plunger head 1231 furthermore has an axial, marginal and distally oriented unlocking pin 1233, by means of which the first locking device 1350 can be unlocked. If the plunger head 1231 is displaced distally, the unlocking pin 1233 comes into contact with the beveled flank of the locking element 1351 and drives the latter radially inwards. As a result, the secondary plunger 1300 is no longer blocked in the distal direction by the locking element 1351, as a result of which the first locking device 1350 is unlocked. The locking element 1351 at the same time serves as a distal stop for the primary plunger 1230.

From the distal region, the secondary plunger 1300 is equipped with an axially oriented second toothed rack 1310 on the inner wall, which toothed rack is more particularly integrally embodied with the secondary plunger 1300 and can interact with the gearwheel 1130 of the housing 1100. In a first state, prior to use of the device 1001, a distal region of the second toothed rack 1310 engages with the gearwheel 1130. Hence the movement of the secondary plunger 1300 is coupled to that of the syringe body 1200, wherein a distal movement of the secondary plunger 1300 is converted into a proximal movement of the syringe body 1200 via the rack-and-pinion gear formed thereby, and with an opposite movement being prevented by the tongue 1114.

In the subsequent paragraphs, the use of the device 1001 for injecting solid matter is now explained in more detail with the aid of FIGS. 6 to 9.

FIG. 6 shows a schematic illustration of an embodiment of the device 1001 according to the invention as per FIG. 6 after the cannula 1220 was inserted into a body 1600, more particularly as a first step when injecting solid matter 1500, before the actuation element 1340 is actuated.

The device 1001 is now grasped by the user on the holding elements 1101 by means of the index and middle finger, and the thumb is placed, initially without pressure, onto the actuation element 1340.

FIG. 7 shows a schematic illustration as per FIG. 6 after the actuation element 1340 was actuated. By actuating the actuation element 1340 in the distal direction by the thumb, the latching elements 1232 are pivoted radially inwards through the unlocking opening 1342, as a result of which the second locking device 1330 is unlocked. Thus the path of the primary plunger 1230 is freed in the distal direction, as a result of which the first spring 1320 relaxes, as a result of which the primary plunger 1230 is displaced relative to the secondary plunger 1300 in the distal direction.

In the syringe body 1200, the distal end of the primary plunger 1230 first of all reaches the third lug 1243 and drives the latter radially outwards by means of the flank between the first section 1234 and the second section 1235. Coupled to this, the elongate section 1240, and hence the lugs 1241 and 1242 as well, are driven radially outwards, as a result of which the path for the solid matter 1500 is freed. Then, driven by the still partly tensioned first spring 1320, the primary plunger 1230 captures the solid matter 1500 with the distal end of the second section 1235 and guides said solid matter into the cannula 1220, more particularly into a region of the cannula 1220 situated outside of the housing 1100. The spacing between the solid matter 1500 and the housing 1100 substantially corresponds to an envisaged implant depth of the solid matter 1500 in the body 1600.

Thus, in this state, a substantial region of the cannula 1220 is in the body 1600, a distal region of the primary plunger 1230 is in the cannula 1220, and hence also in the body 1600, and the solid matter 1500 is within the body 1600 in front of the distal end of the primary plunger 1230.

Concurrent with the complete insertion of the primary plunger 1230, the plunger head 1231 contacts the locking element 1351 of the first locking device 1350 with the unlocking pin 1233, with the beveled flank of the locking element 1351 being driven radially inwards. Hence the first locking device 1350 is unlocked, and so the secondary plunger 1300 can be displaced axially within the housing 1100. In this state, the syringe body 1200 is distanced from the plunger head 1231. The primary plunger 1230 is blocked in the proximal direction by the now only slightly tensioned spring 1320 and in the distal direction by the locking element 1351, and hence it is blocked on both sides.

If the secondary plunger 1300 is now inserted into the housing 1100 in the distal direction by pressure exerted on the actuation element 1340, a proximal movement, in the direction of the plunger head 1231 along the primary plunger 1230, of the syringe body 1200, and hence the cannula 1220 as well, is brought about at the same time via the first and the second toothed rack 1210, 1310, which are coupled via the gearwheel 1130; at the same time the primary plunger 1230 remains fixed in its position. As a result, the cannula 1220 is withdrawn back over the solid matter 1500 and the region of the primary plunger 1230 protruding out of the housing 1100, and so the solid matter 1500 remains outside.

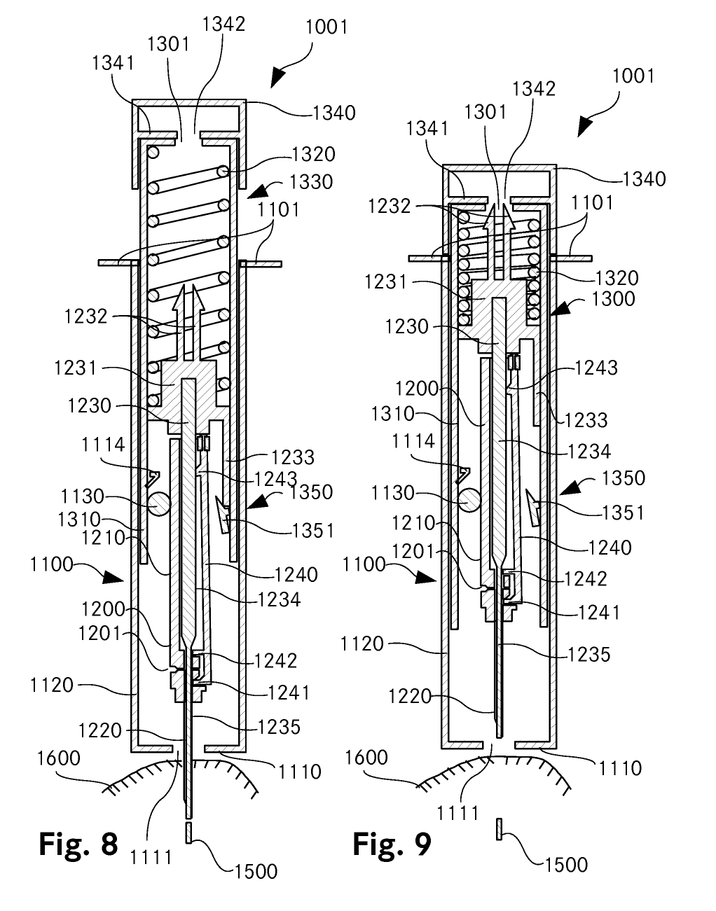

FIG. 8 shows a schematic illustration as per FIG. 7 after the partial insertion of the secondary plunger 1300. The distal end of the cannula 1220 is approximately flush with the distal end of the primary plunger 1230. In this state, the syringe body 1200 contacts the plunger head 1231. When the secondary plunger 1300 is inserted further by means of the actuation of the actuation element 1340 in the distal direction, the primary plunger 1230 is proximally displaced via the plunger head 1231 and into the housing 1100 through the syringe body 1200, and hence together with the cannula 1220, as a result of which the first spring 1320 is partially recompressed.

So that the secondary plunger 1300 cannot be displaced in the proximal direction by the first spring 1320 when the former is released, provision is made for the tongue 1114, which interacts with the second toothed rack 1310 and so prevents the secondary plunger 1300 from returning into the body 1600, or prevents distal displacement of the cannula 1220 back into said body.

FIG. 9 shows a schematic illustration as per FIG. 8 after the injection has been carried out, with the cannula 1220 and the distal region of the primary plunger 1230 lying completely within the housing 1100.

The cannula 1220 and the first spring 1320 consist of metal, more particularly of steel. The remaining parts of the device 1001 are made of a plastic, for example, polymethyl methacrylate (PMMA) or polycarbonate. A person skilled in the art also knows of further suitable materials that can be used for individual parts of the device 1001. By way of example, the first spring 1320 could also be made of a plastic.

The holding elements 1101 can also be designed as a circular disk encircling the housing or as further holding elements 1101 known to a person skilled in the art. In particular, provision can also be made for an ergonomically adapted shape of the holding elements 1101.

In summary, it should be noted that the invention develops a device for injecting solid matter, which is very easy to handle and can also be used effortlessly and intuitively by novice users.

1. Device (1; 1001) for injecting solid matter (500; 1500) into a human or animal body (600; 1600), more particularly a syringe for injecting a solid-matter medicament, comprising:
a) a housing (100; 1100);
b) a syringe body (200; 1200) for holding the solid matter (500; 1500), connected to a cannula (220; 1220);
c) a primary plunger (230; 1230), which can be displaced in the syringe body (200; 1200) and in the cannula (220; 1220);
d) a secondary plunger (300; 1300), which can be displaced in the housing (100; 1100);
e) and comprising a force-transmission mechanism, which can act between the secondary plunger (300; 1300) and the syringe body (200; 1200) and/or the cannula (220; 1220); wherein
f) an actuation of the secondary plunger (300; 1300) in the distal direction can bring about a displacement of the syringe body (200; 1200) and/or the cannula (220; 1220) in the proximal direction,
characterized in that
g) the device (1; 1001) comprises a first energy store, wherein energy from the first energy store can be used for the distal displacement of the primary plunger (230; 1230).

2. Device (1; 1001) according to claim 1, characterized in that the force-transmission mechanism comprises a rack-and-pinion gear.

3. Device (1; 1001) according to claim 1, characterized in that the force-transmission mechanism is embodied as a rack-and-pinion gear, which comprises a first toothed rack (210; 1210), a second toothed rack (310; 1310) and a gearwheel (130; 1130), wherein
a) the gearwheel (130; 1130) is mounted in the housing (100; 1100) such that it can rotate freely; and
b) the first toothed rack (210; 1210) is connected to the syringe body (200; 1200); and
c) the second toothed rack (310; 1310) is connected to the secondary plunger (300; 1300); wherein
d) the gearwheel (130; 1130) interacts with the first and the second toothed rack (210, 310; 1210, 1310).

4. Device (1; 1001) according to claim 1, characterized in that it comprises a first locking device (350; 1350), by means of which the secondary plunger (300; 1300) can be blocked in respect of an axial displaceability in the housing (100; 1100).

5. Device (1; 1001) according to claim 4, characterized in that the first locking device (350; 1350) can be deactivated by the primary plunger (230; 1230), more particularly by a distal displacement of the primary plunger (230; 1230).

6. Device (1; 1001) according to claim 1, characterized in that the first energy store comprises a first spring (320; 1320), in particular for displacing the primary plunger (230; 1230) in the distal direction, wherein the first spring (320; 1320) is more particularly embodied as a helical spring.

7. Device (1; 1001) according to claim 1, characterized in that the primary plunger (230; 1230) is guided in the secondary plunger (300; 1300).

8. Device (1; 1001) according to claim 6, characterized in that the first spring (320, 1320) is arranged in the secondary plunger (300; 1300) such that the primary plunger (230; 1230) can be axially displaced from the secondary plunger (300; 1300) by a spring force from the first spring (320; 1320).

9. Device (1; 1001) according to claim 6, characterized in that it comprises a second locking device (330; 1330), by means of which the primary plunger (230; 1230) can be locked with respect to the secondary plunger (300; 1300), particularly if the first spring (320; 1320) is tensioned.

10. Device (1; 1001) according to claim 9, characterized in that the secondary plunger (300; 1300) comprises an actuation element (340; 1340), by means of which the second locking device (330; 1330) can be unlocked manually, wherein the actuation element (340; 1340) in particular is arranged on a proximal end of the secondary plunger (300; 1300) and preferably embodied as a pushbutton.

11. Device (1001) according to claim 9, characterized in that the primary plunger (1230) is connected to axially and proximally oriented latching elements (1232), which can be latched into an opening arranged distally in the secondary plunger (1300) and can be unlocked by the actuation element (1340).

12. Device (1; 1001) according to claim 1, characterized in that the syringe body (200; 1200) comprises a retention device for retaining the solid matter (500; 1500) before the injection.

13. Device (1001) according to claim 1, characterized in that, in one state, more particularly after the device (1001) has been used, the cannula (1220) and a distal end of the primary plunger (1230) are situated entirely within the housing (1100).

14. Device (1001) according to claim 1, characterized in that, in a second state, in which the secondary plunger (1300) is partly inserted, the cannula (1220) is withdrawn proximally, and so the solid matter (1500) is not held in the cannula (1220) and the primary plunger (1230) is at best partly held by the cannula (1220), or, in a third state, when the secondary plunger (1300) is completely inserted, more particularly after the device (1001) has been used, the primary plunger (1300) and the cannula (1220) are withdrawn together with respect to the first state.

15. Device (1001) according to claim 1, characterized in that the primary plunger (1230) comprises a plunger head (1231) at a proximal end, wherein the plunger head (1231) is distanced from the syringe body (1200) in a first state, when the spring (1320) is relaxed before the secondary plunger (1300) is inserted, and the syringe body (1200) contacts the plunger head (1231) in the second state.

16. Device (1001) according claim 1, characterized in that the primary plunger (1230) comprises a first, proximal section (1234) and a second, distal section (1235), wherein the first section (1234) has a larger diameter than the second section (1235).

17. Device (1; 1001) according to claim 2, characterized in that the force-transmission mechanism is embodied as a rack-and-pinion gear, which comprises a first toothed rack (210; 1210), a second toothed rack (310; 1310) and a gearwheel (130; 1130), wherein
a) the gearwheel (130; 1130) is mounted in the housing (100; 1100) such that it can rotate freely; and
b) the first toothed rack (210; 1210) is connected to the syringe body (200; 1200); and
c) the second toothed rack (310; 1310) is connected to the secondary plunger (300; 1300); wherein
d) the gearwheel (130; 1130) interacts with the first and the second toothed rack (210, 310; 1210, 1310).

18. Device (1; 1001) according to claim 2, characterized in that it comprises a first locking device (350; 1350), by means of which the secondary plunger (300; 1300) can be blocked in respect of an axial displaceability in the housing (100; 1100).

19. Device (1; 1001) according to claim 3, characterized in that it comprises a first locking device (350; 1350), by means of which the secondary plunger (300; 1300) can be blocked in respect of an axial displaceability in the housing (100; 1100).

20. Device (1; 1001) according to claim 2, characterized in that the first energy store comprises a first spring (320; 1320), in particular for displacing the primary plunger (230; 1230) in the distal direction, wherein the first spring (320; 1320) is more particularly embodied as a helical spring.
